(19) United States
(12) Patent Application Publication
(10) Pub. No.: US 2008/0166407 A1
(43) Pub. Date: Jul. 10, 2008

(54) Solid oral formulations for combination therapy

(76) Inventors: Shalaby W. Shalaby, Anderson, SC (US); Kenneth David Gray, Clemson, SC (US); Joel T. Corbett, Seneca, SC (US)

Correspondence Address: LEIGH P. GREGORY, PO BOX 168, CLEMSON, SC, 29633-0168, US

(21) Appl. No.: 12/072,083

(22) Filed: Feb. 21, 2008

Related U.S. Application Data
(63) Continuation-in-part of application No. 11/494,662, filed on Jul. 27, 2006.
(60) Provisional application No. 60/704,018, filed on Jul. 29, 2005.

(51) Int. Cl. A61K 9/24; A61K 9/36; A61K 9/20; A61K 31/341; A61K 31/192; A61K 31/60; A61K 31/19; A61K 31/4439; A61K 31/167

(52) U.S. Cl. 424/465; 424/480; 514/471; 514/569; 514/570; 514/338; 514/629; 424/472; 514/165

(57) ABSTRACT

Multifunctional, single, bilayer, and trilayer coated tablets for combination therapy are formed wherein the bioactive agents responsible for the therapeutic multifunctionality are present as a combination of a gastric acid-reducing agent, such as omeprazole and ranitidine, and at least one analgesic/anti-inflammatory agent, such as acetaminophen, naproxen sodium, ibuprofen, tolmetin, and aspirin.

The present invention is a continuation in part of U.S. patent application Ser. No. 11/494,662 filed on Jul. 27, 2006, which claims the benefit of prior provisional application, U.S. Ser. No. 60/704,018 filed on Jul. 29, 2005.

### FIELD OF THE INVENTION

### Background of the Invention

The parent application of this invention related to (1) pharmaceutical compositions that are solid oral formulations for combination therapy comprising an extended release acetaminophen as the principal analgesic agent admixed with a second compound as a principal anti-inflammatory agent with concomitant antipyretic and analgesic properties, plus a third component as a principal agent for reducing gastric acid secretion; (2) novel pharmaceutical formulations for combination therapy comprising an anti-inflammatory agent and a second agent for reducing gastric acid secretion; and (3) a novel bilayered tablet for the controlled release of three active agents—an analgesic agent, comprising acetaminophen in one disc (or layer) adjoined with a second disc comprising a non-steroidal, anti-inflammatory drug and an agent for reducing gastric acid secretion. To further the teaching of the parent invention, the instant invention provides an additional means to modulate the drug release of the acetaminophen systems containing a total of three active agents. Recognition of the clinical importance of analgesic drugs, such as aspirin, and interest in addressing their unexplored co-administration with agents for reducing gastric acid secretion, led the present inventors to the pursuit of such an approach to develop different forms of mixed formulations for combined therapy using conventional and novel methods for tablet preparation.

### SUMMARY OF THE INVENTION

This invention is generally directed to a combination therapy coated tablet comprising (a) a therapeutically effective dose of a non-steroidal anti-inflammatory drug (NSAID); (b) a therapeutically effective dose of an agent for reducing gastric acid secretion; (c) a thermoplastic hydrophilic polymer; (d) a microparticular excipient; and (e) a cellulosic enteric coating, wherein the NSAID is selected from the group consisting of naproxen sodium, ibuprofen, and tolmetin, the agent for reducing gastric acid secretion is ranitidine, the thermoplastic hydrophilic polymer is a solid polyethylene glycol, the microparticular excipient is microcrystalline cellulose, and the enteric coating is hydroxypropyl cellulose.

A specific aspect of this invention deals with a combination therapy coated tablet comprising (a) a therapeutically effective dose of an analgesic drug; (b) a therapeutically effective dose of an agent for reducing gastric acid secretion; (c) a thermoplastic hydrophilic polymer; (d) a microparticular excipient; and (e) a cellulosic enteric coating, wherein the analgesic drug is aspirin, the agent for reducing gastric acid secretion is omeprazole, the thermoplastic hydrophilic polymer is a solid polyethylene glycol, the microparticular excipient is microcrystalline cellulose, and the enteric coating is hydroxypropyl methyl cellulose phthalate.

Another specific aspect of the instant invention deals with a combination therapy coated bilayer tablet consisting of one layer for the delayed release of acetaminophen and a second layer for the fast release of a therapeutically effective dose of a non-steroidal anti-inflammatory drug and a therapeutically effective dose of an agent for reducing gastric acid secretion wherein the acetaminophen is predispersed in a molten water-soluble thermoplastic polymer prior to a 2-step preparation of the tablet, the entire tablet is coated with a polymeric material to provide the needed physicomechanical properties for achieving the intended mode of release of the three agents. Meanwhile, the general method for the preparation of the bilayer tablet consists of the steps of (a) dispersing/dissolving the acetaminophen (AMP) in molten polyethylene glycol (PEG); (b) cooling the AMP/PEG system and grinding; (c) pressing the ground AMP/PEG system at room temperature and a pressure of less than 500 lbs; (d) opening the mold and placing a mixture of the two drugs intended for fast-release over the formed AMP/PEG first layer; (e) pressing the combined system at room temperature and a pressure of 500 to 1000 lbs; and (f) removing the bilayer tablet and dip-coating it in a solution of hydroxypropyl cellulose in 2-propanol followed by air drying.

A special aspect of this present invention deals with a combination therapy coated bilayered tablet consisting of one layer comprising a therapeutically effective dose of an analgesic drug and a second layer comprising a therapeutically effective dose of an agent for reducing gastric acid secretion wherein each layer, independently, contains microparticular and hydrophilic polymeric excipients, and wherein the analgesic drug is aspirin, the acid reducing agent is omeprazole, the hydrophilic polymeric excipient is a solid polyethylene glycol, and the microparticular excipient is microcrystalline cellulose, the entire tablet is coated with a hydroxypropyl methyl cellulose phthalate.

Another special aspect of the invention deals with a combination therapy coated trilayer tablet consisting of one layer comprising a therapeutically effective dose of an analgesic drug, a microparticular and hydrophilic polymeric excipients, a second layer comprising a mixture of a microparticular and hydrophilic polymeric excipients, and a therapeutically effective dose of an agent for reducing gastric acid secretion, and a third drug-free layer of a solid, water-soluble polymer adjoining the first and second drug-containing layers, wherein the analgesic drug is aspirin, the agent for reducing gastric acid secretion is omeprazole, the microparticular excipient is microcrystalline cellulose, the hydrophilic polymeric excipient is a polyethylene glycol solid, and the water-soluble polymer is a polyethylene glycol; the entire tablet is coated with a hydroxypropyl methyl cellulose phthalate, and wherein the agent for reducing gastric acid secretion is omeprazole in the form of its magnesium salt.

A key aspect of the invention deals with a combination therapy coated trilayer tablet consisting of one layer comprising a therapeutically effective dose of an analgesic drug mixed with microparticulate and hydrophilic polymeric excipients, a second layer comprising a mixture of microparticular and hydrophilic polymeric excipients, an inorganic base and a therapeutically effective dose of an agent for reducing gastric acid secretion and a third drug-free layer of a solid water-soluble polymer adjoining the first and second drug-containing layers, wherein the analgesic drug is aspirin, the agent for reducing acid secretion is omeprazole, the microparticular excipient is microcrystalline cellulose, the hydrophilic excipient is a polyethylene glycol, the solid water-soluble polymer is a polyethylene glycol, and the inorganic base is sodium bicarbonate; the entire tablet is coated with a hydroxypropyl methyl cellulose phthalate.

### DETAILED DESCRIPTION OF PREFERRED EMBODIMENTS

This invention relates to new, solid formulations for combination therapy for the directed release and/or controlled release of at least two bioactive agents from single, bilayer, or trilayer, enterically coated tablets. The combinations of the bioactive agents are selected such that one of the agents mediates possible undesirable side effects of the other agent or agents, as in the case of using an agent for reducing gastric acid secretion in combination with an acid-producing drug of different pharmacological activities. This is to minimize the effect of in situ generated acidity on the cell lining of the gastrointestinal tract and patient discomfort by reducing the acidic gastric secretion.

A general aspect of the instant invention is the use of (1) a thermoplastic hydrophilic polymer, such as a solid polyethylene glycol, having a molecular weight of more than 1 kDa, as a water soluble excipient to facilitate a rapid release of the bioactive agent(s) for reducing gastric acid secretion; (2) a microparticular binder, as microcrystalline cellulose, to aid the tableting process of the mixed, solid formulation and maximize the uniformity of the active agent distribution therein; and (3) a cellulosic enteric coating to modulate the release of the active agent(s) and prevent premature disintegration of the mixed tablet formulation.

A specific aspect of this invention deals with bifunctional coated tablets comprising (1) ranitidine or omeprazole as the agent for reducing gastric acid secretion; (2) naproxen sodium, ibuprofen, or aspirin as the analgesic/anti-inflammatory agent; and (3) hydroxypropyl cellulose or hydroxypropyl methyl cellulose phthalate as a hydrophilic or relatively hydrophobic enteric coating, respectively—a fast release of the bioactive coating can be achieved using the hydrophilic coating, while increasing the coating thickness can be used to slow down the drug release if so needed.

Another specific aspect of the present invention deals with a multifunctional bilayer tablet wherein (1) one layer comprises ranitidine or omeprazole as the agent for reducing gastric acid secretion; (2) a second layer contains acetaminophen and/or naproxen sodium as the analgesic/anti-inflammatory agent; and (3) hydroxypropyl cellulose is used as the hydrophilic enteric coating. Alternatively, the combination therapy bilayer tablet comprises (1) aspirin as the analgesic agent; (2) ranitidine or omeprazole as the agent for reducing gastric acid secretion; and (3) hydroxypropyl methyl cellulose phthalate or hydroxypropyl cellulose as the enteric coating.

A special aspect of this invention pertains to the multifunctional trilayer coated tablet comprising (1) one aspirin-containing layer; (2) a second layer containing omeprazole and/or ranitidine as the agent for reducing gastric acid secretion; (3) a drug-free layer adjoining the two drug-containing layers; and (4) hydroxypropyl methyl cellulose phthalate as the enteric coating. Further illustrations of the present invention are provided by the following examples:

### Example 1

### Preparation of a Multi-Drug Component, Single-Layer Tablet: General Method

The multi-drug component tablet consisted of two drug components in addition to excipients that accelerate the release the drugs into the intestine over a desired time frame (approximately 4 hours). The drug components include (1) an NSAID or analgesic drug, and (2) an enteric acid blocker. In the actual production of the tablet, all chemical components were weighed individually and ground together using a mortar and pestle. The ground material was added to a tablet mold which is pressed to form the final tablet. The edges of the tablet were deburred and the tablet weight was recorded. Tablets were stored in glass vials that were exposed to a vacuum atmosphere, purged with nitrogen, and taped.

### Example 2

### Preparation of a Multi-Drug, Single-Layer Tablet Following Example 1: Specific Method 1-A

The tablet consisted of (1) three drugs, namely, acetaminophen, naproxen sodium, and ranitidine hydrochloride; (2) polyethylene glycol-20 (PEG-20, molecular weight=20 kDa) as a hydrophilic thermoplastic excipient; and (3) microcrystalline cellulose as a binder. The individual components of the tablet were weighed, thoroughly mixed, and ground using a mortar and pestle. The ground mixture was transferred to a tablet mold, which was pressed using 10,000 lbs pressure. After releasing the pressure on the mold, the tablet was removed, the edges were deburred, and the final tablet weight was recorded. The tablet was then placed in a clean vial, which was subjected to vacuum and purged with nitrogen before the lid was sealed for storage prior to coating.

### Example 3

### Preparation of a Multi-Drug Component, Single-Layer Tablet Following Example 1: Specific Method 1-B

The tablet consisted of four different chemical compounds: naproxen sodium, ranitidine hydrochloride, polyethylene glycol (20,000 molecular weight), and microcrystalline cellulose. The individual components of the tablet were weighed and ground together using a mortar and pestle. The resulting ground material was added to a tablet mold which is pressed with 10,000 lbs pressure. After reducing the pressure on the mold, the tablet was removed, the edges were deburred, and the final tablet weight was recorded. The tablet was then placed into a clean vial, which was subjected to vacuum and purged with nitrogen before the lid was sealed for storage prior to coating.

### Example 4

### Preparation of a Multi-Drug, Single-Layer Component Tablet Following Example 1: Specific Method 1-C

The tablet consisted of four different chemical compounds: ibuprofen, ranitidine hydrochloride, polyethylene glycol (20,000 M_{w}), and microcrystalline cellulose. The individual components of the tablet were weighed and ground together using a mortar and pestle. The resulting ground material was added to a tablet mold which was pressed with 10,000 lbs pressure. After reducing the pressure on the mold, the tablet was removed, the edges were deburred, and the final tablet weight was recorded. The tablet was then placed into a clean vial, which was subjected to vacuum and purged with nitrogen before the lid was sealed for storage prior to coating.

### Example 5

### Preparation of a Multi-Drug, Single-Layer Component Tablet Following Example 1: Specific Method 1-D

The tablet consisted of four different chemical compounds: aspirin (as an analgesic), omeprazole, polyethylene glycol (20,000 M_{w}), and microcrystalline cellulose. The individual components of the tablet were weighed and ground together using a mortar and pestle. The resulting ground material was added to a tablet mold which was pressed with 10,000 lbs pressure. After reducing the pressure on the mold, the tablet was removed, the edges were deburred, and the final tablet weight was recorded. The tablet was then placed into a clean vial, which was subjected to vacuum and purged with nitrogen before the lid was sealed for storage prior to coating.

### Example 6

### Preparation of Multi-Drug Bilayer Tablet: General Method

A bilayer tablet consisted of a slow-releasing base layer as well as a fast-releasing layer. Drugs in the slow-releasing layer were designed to release from the tablet over an extended time period (i.e. 8 hours), whereas drugs in the fast-releasing layer were designed to release over a shorter time period (approximately 4 hours). Furthermore, the slow-releasing layer was composed of an NSAID in combination with high molecular weight polyethylene glycol (PEG) that controls the release of the NSAID over time. The materials for the base layer were prepared by combining the PEG and NSAID and grinding the materials together using a mortar and pestle, followed by heating the ground materials in a glass vial at 70° C. for 30 minutes. The heated material was removed from the vial with a spatula, allowed to cool to room temperature, then ground using a mortar and pestle into a form that was appropriate for future use. During tablet production, the prepared PEG/NSAID material was added to a tablet mold and pressed quickly with a small load of pressure. Subsequently, the materials for the fast-release layer were weighed, ground using a mortar and pestle, added to the tablet mold on top of the base layer, and pressed with 5,000 lbs, 7,500 lbs, and finally 10,000 lbs pressure. The tablet was then removed from the mold and the edges were cleaned of excess material; weight was recorded and the tablet was placed in a vial for storage prior to coating.

### Example 7

### Preparation of Multi-Drug Bilayer Tablet Following Example 6: Specific Method 6-A

The tablet consisted of two layers: an acetaminophen-releasing base layer and a fast-releasing naproxen sodium/ranitidine layer (Table I). The base layer consisted of acetaminophen and polyethylene glycol 35,000 in equal mass ratios of 325 mg; the two components were combined in a glass vial, heated at 70° C. for 30 minutes, poured onto a Teflon sheet and allowed to cool to room temperature, then ground using a mortar and pestle. Approximately 650 mg of the acetaminophen-releasing base layer was added to the tablet mold and pressed with 1,000 lbs pressure prior to adding the components of the fast-release layer. Following the initial light compression of the base layer, the naproxen sodium, ranitidine, and polyethylene glycol 20,000 were weighed individually and ground together using a mortar and pestle. The resulting ground material was weighed and recorded, and the material was added to the tablet mold above the acetaminophen base layer. Subsequently, the tablet mold was pressed with 5,000 lbs, 7,500 lbs, and 10,000 lbs pressure in succession in order to completely form the final tablet (pressures are applied for at least one minute prior to increasing pressure on the mold or removing the mold from the pressure load altogether). After finally reducing the pressure on the mold, the tablet was removed, the edges were deburred, and the final tablet weight was recorded. The tablet was then placed into a clean vial, which was subjected to vacuum and purged with nitrogen before the lid was sealed for storage.

### Example 8

### Preparation of Multi-Drug Bilayer Tablet Following Example 6: Specific Method 6-B

The tablet consisted of two layers: an acetaminophen-releasing base layer and a fast-releasing ibuprofen/ranitidine layer (Table I). The base layer consisted of acetaminophen and polyethylene glycol 35,000 in equal mass ratios of 325 mg; the two components were combined in a glass vial, heated at 70° C. for 30 minutes, poured onto a Teflon sheet and allowed to cool to room temperature, then ground using a mortar and pestle. Approximately 650 mg of the acetaminophen-releasing base layer was added to the tablet mold and pressed with 1,000 lbs pressure prior to adding the components of the fast-release layer. Following the initial light compression of the base layer, the naproxen sodium, ranitidine, and polyethylene glycol 20,000 were weighed individually and ground together using a mortar and pestle. The resulting ground material was weighed and recorded, and the material was added to the tablet mold above the acetaminophen base layer. Subsequently, the tablet mold was pressed with 5,000 lbs, 7,500 lbs, and 10,000 lbs pressure in succession in order to completely form the final tablet (pressures are applied for at least one minute prior to increasing pressure on the mold or removing the mold from the pressure load altogether). After finally reducing the pressure on the mold, the tablet was removed, the edges were deburred, and the final tablet weight was recorded. The tablet was then placed into a clean vial, which was subjected to vacuum and purged with nitrogen before the lid was sealed for storage.

### Example 9

### Preparation of a Multi-Drug, Bilayer Tablet Following Example 6: Specific Method 6-C

A different type of bilayer tablet was prepared with one layer containing an analgesic drug (aspirin) and another layer containing an enteric acid blocker (omeprazole). Each layer also contains polyethylene glycol (20,000 M_{w}) and microcrystalline cellulose. The first step in preparation of the tablet was to weigh out the individual components of the aspirin layer. Subsequently, the weighed material was ground together using a mortar and pestle, and the ground material was pressed in the tablet mold with less than 5,000 lbs. pressure. Next, the components of the omeprazole layer were weighed out and ground together. The ground material was added to the tablet mold on top of the aspirin layer, and the mold was compressed with 10,000 lbs. pressure to form the final bilayer tablet.

### Example 10

### Preparation of a Two-Drug, Trilayer Tablet: General Method

The trilayer tablet consisted of two layers separately containing aspirin or omeprazole, and separated by a middle layer of PEG (M_{w}=20 kDa). The components of the aspirin layer were weighed, ground using a mortar and pestle, and compressed with less than 5,000 lbs. pressure. Then PEG was added to the tablet mold above the aspirin-containing layer, and the mold was compressed a second time with less than 5,000 lbs. pressure. Lastly, the components of the omeprazole layer were weighed out, ground, and added to the tablet mold above the other two layers. The mold was compressed with 10,000 lbs pressure to form the final tablet.

### Example 11

### Preparation of a Multi-Drug, Trilayer Tablet Following Example 10: Specific Method 10-A

A tablet was prepared following the general method outlined in Example 10. The final tablet was coated with hydroxypropyl methyl cellulose phthalate.

### Example 12

### Preparation of a Multi-Drug, Trilayer Tablet Following Example 10: Specific Method 10-B

A second type of trilayer tablet was prepared following the general method outlined in Example 10, and the magnesium salt of omeprazole was used in making the tablet instead of the free base form of the drug. The final tablet was coated with hydroxypropyl methyl cellulose phthalate.

### Example 13

### Preparation of a Multi-Drug, Trilayer Tablet Following Example 10: Specific Method 10-C

A third type of trilayer tablet was prepared following the general method outlined in Example 10, and an inorganic base (sodium bicarbonate) was included in the layer containing the agent for reducing gastric acid secretion. The final tablet was coated with hydroxypropyl methyl cellulose phthalate.

### Example 14

### Tablet Coating with Hydroxypropyl Cellulose: General Method

Tablets were coated by dipping in a 5% solution of hydroxypropyl cellulose (HPC) in isopropyl alcohol. Multiple dipping steps were necessary to achieve the desired add-on weight of the coating layer which functions, in part, to protect tablets during handling.

### Example 15

### Tablet Coating with Hydroxypropyl Methyl Cellulose Phthalate: General Method

Tablets were coated by dipping in a 5% solution of hydroxypropyl methyl cellulose phthalate (HPC) in a solvent mixture of isopropyl alcohol and acetone. Multiple dipping steps were necessary to achieve the desired add-on weight of the coating layer which, in part, functions to protect tablets during handling.

### Example 16

### General Method for Monitoring the Drug Release Profile(s) from Multi-Drug Component Tablets

### Drug Release Studies

Release studies were conducted in order to determine/verify the release of individual drug components from the multicomponent tablets. These studies were carried out over eight hour time periods at 37° C., and samples were collected at one hour intervals for analysis by HPLC. The first hour of the release study involved incubating the tablet in 10 milliliters of simulated gastric fluid (pH=1.2). In the seven subsequent hours, the tablets were incubated in 10 milliliters of deionized water. Incubation fluid was filtered into clean vials after each hour using 45 micrometer syringe filters and disposable 10 milliliter syringes, and fresh deionized water was added to the tablet before returning it to 37° C. The eight individual filtrates provided undiluted samples for HPLC analysis, and from those samples eight additional 100× diluted samples were produced. One milliliter of undiluted filtrate was added by pipette to a 100 milliliter volumetric flask, which was then filled to 100 milliliters with deionized water. The diluted solution was mixed by covering the flask with parafilm and shaking vigorously for approximately 15 seconds. The undiluted and diluted samples were then added to HPLC tubes and analyzed by reverse phase chromatography using water (1% trifluoroacetic acid) and acetonitrile (1% trifluoroacetic acid) as solvents.

### High Pressure Liquid Chromatography (HPLC) Analysis of Released Drugs

HPLC analysis utilized 22 minute run times per sample where the acetonitrile gradient was increased from 5% to 95%. This was followed by a 10 minute equilibration period in which the solvent gradients were allowed to return to 95% water and 5% acetonitrile. Undiluted samples were injected onto the column in volumes between 1 and 5 microliters, whereas the diluted samples were injected in volumes ranging from 5 to 30 microliters.

### Example 17

### Drug Release Profiles of a Typical Three-Drug Component, Single-layer Tablet Containing Acetaminophen, Ranitidine Hydrochloride and Naproxen Sodium (Tablet I-A)

The tablet was prepared as per the method of Example 2 using 325 mg of acetaminophen, 325 mg of PEG (M_{w}=35 kDa), 110 mg of naproxen sodium, and 37.5 mg of ranitidine hydrochloride, and the tablet was coated with hydroxypropyl cellulose following the method of Example 14. The general method of determining the drug release profile described in Example 16 was used to monitor separately the release profile of acetaminophen, naproxen sodium and ranitidine. The respective results are summarized in Table I.

TABLE I
Release Data of Three-drug Component, Single-layer Tablet, I-A*

| | | |
|---|---|---|
| Acetaminophen | Naproxen Sodium | Ranitidine |

| Hour | % Release | Hour | % Release | Hour | % Release |
|---|---|---|---|---|---|
| 1 | 7.48 | 1 | 0.18 | 1 | 3.33 |
| 2 | 18.90 | 2 | 32.92 | 2 | 47.12 |
| 4 | 38.04 | 4 | 54.80 | 4 | 73.83 |
| 6 | 54.08 | 6 | 58.17 | 6 | 78.57 |
| 8 | 70.03 | 8 | 59.03 | 8 | 79.59 |

*Coated with 1.476% hydroxypropyl cellulose; data are based on triplicate runs.

### Example 18

### Drug Release from a Typical Two-Drug Component, Single-Layer Tablet Containing Naproxen Sodium and Ranitidine HCl (Tablet I-B)

The tablet was prepared as per the method of Example 3 using 100 mg of naproxen sodium, 37.5 mg of ranitidine hydrochloride, and polyethylene glycol having a molecular weight of 20 kDa (PEG-20) as and polyethylene glycol having a molecular weight of 20 kDa (PEG-20) as a hydrophilic excipient (600 mg) and microcrystalline cellulose (MCC, 37 mg) as a binder. The tablet was coated with hydroxypropyl cellulose following the method of Example 14. The release profile of the individual drug was determined using the general method of Example 16 and the results are summarized in Table II.

TABLE II
Release Data of Two-drug Component, Single-layer Tablet I-B*

| Naproxen Sodium | | | | Ranitidine | | | |
|---|---|---|---|---|---|---|---|
| Hour | % Release | | | Hour | % Release | | |
| 1 | 0.29 | 0.34 | 0.32 | 1 | 12.06 | 17.91 | 16.41 |
| 2 | 13.85 | 0.77 | 1.45 | 2 | 40.83 | 33.69 | 30.22 |
| 3 | 50.78 | 21.09 | 28.37 | 3 | 89.66 | 60.48 | 65.83 |
| 4 | 55.82 | 47.86 | 52.85 | 4 | 95.39 | 95.52 | 94.20 |
| 5 | 56.57 | 50.68 | 58.81 | 5 | 96.14 | 98.82 | 101.01 |
| 6 | 56.98 | 51.29 | 59.79 | 6 | 96.23 | 99.24 | 101.81 |
| 7 | 57.28 | 51.63 | 60.31 | 7 | 96.30 | 99.33 | 101.93 |
| 8 | 57.61 | 51.94 | 60.71 | 8 | 96.35 | 99.39 | 102.22 |

*Tablet weight = 759.6 ± 3.4 mg. Coated with 1.66 ± 0.02% hydroxypropyl cellulose; data are based on triplicate runs.

### Example 19

### Drug Release from a Typical Two-Drug Component, Single-layer Tablet Containing Ibuprofen and Ranitidine HCl (Tablet I-C)

The tablet was prepared as per the method of Example 4 using polyethylene glycol having a molecular weight of 20 kDa (PEG-20) as a hydrophilic excipient and microcrystalline cellulose (MCC) as a binder. The tablet was coated with hydroxypropyl cellulose as per Example 14. Specific details of the tablet production and coating schemes are summarized as follows.

### Tablet Production

The tablet contained a fast-releasing ibuprofen/ranitidine layer that also contained polyethylene glycol having a molecular weight of 20 kDa (PEG-20) and microcrystalline cellulose (MCC). Ibuprofen made up approximately 14% of the tablet with 110 milligrams, and ranitidine made up approximately 4.8% of the tablet with 37.5 milligrams; PEG-20 and MCC account for 76.5% and 4.7% of the total tablet weight, respectively. The individual tablet materials were weighed out and ground together using a mortar and pestle. The weight of the resulting ground material was determined and subsequently added to the tablet mold. Pressure of 10,000 lbs. was applied to the mold in order to press the tablet into its final form. The pressure was then released and the tablet was removed from its mold. The edges of the tablet were deburred and a final weight was recorded. The tablet was placed in a vial that was subjected to vacuum, purged with nitrogen, and sealed for storage.

Coating of Tablets with Hydroxypropyl Cellulose

All tablets were coated by dipping in a 5% solution of hydroxypropyl cellulose (HPC) in isopropyl alcohol. Tablets were held with forceps and dipped by hand into the coating solution. Multiple dips were required to achieve approximately 1.5% coating, and between each dip the tablet was allowed to dry for at least 30 minutes in front of a fan under a fume hood.

The drug release studies were conducted as per Example 16 and the corresponding results are summarized in Table III.

TABLE III
Release Data of Two-drug Component, Single-layer Tablet I-C*

| Ibuprofen | | | | | Ranitidine | | | | |
|---|---|---|---|---|---|---|---|---|---|
| Hour | % Release | | | Avg | Hour | % Release | | | Avg |
| 1 | 0.68 | 0.20 | 0.15 | 0.34 | 1 | 6.28 | 3.79 | 3.21 | 4.43 |
| 2 | 11.64 | 14.87 | 7.89 | 11.47 | 2 | 21.40 | 25.65 | 16.05 | 21.03 |
| 3 | 23.05 | 23.15 | 25.59 | 23.93 | 3 | 35.32 | 35.12 | 34.97 | 35.14 |
| 4 | 23.84 | 25.69 | 32.19 | 27.24 | 4 | 36.07 | 38.97 | 46.11 | 40.38 |

*Coating add-on = 1.29%; data are based on triplicate runs.

### Example 20

### Drug Release Profile of a Typical Three-Drug Component, Two-Layer Tablet Containing Acetaminophen, Ranitidine HCl and Naproxen Sodium with PEG-20 as an Excipient (Tablet II-A)

The tablet was prepared as per the method of Example 7 and coated with hydroxypropyl cellulose as per Example 14. The specifics of the tablet production are summarized below.

### Tablet Production

The tablet consisted of two layers: an acetaminophen-releasing base layer and a fast-releasing naproxen sodium/ranitidine layer. The base layer consisted of acetaminophen (325 mg) and polyethylene glycol 35 kDa (325 mg) in equal ratios; the two components were combined in a glass vial, heated at 80° C. for 30 minutes, and then the molten mixture was poured onto a Teflon sheet and collected into a separate vial for future use. Approximately 650 mg of the acetaminophen base layer was added to the tablet mold and pressed with 1,000 lbs of pressure prior to adding the components of the fast-release layer. Following the initial light compression of the base layer, the naproxen sodium, ranitidine, and polyethylene glycol 20 kDa were weighed individually and ground together using a mortar and pestle. The resulting ground material was weighed and added to the tablet mold above the acetaminophen base layer. Subsequently, the tablet mold was pressed with 5,000 lbs, 7,500 lbs, and 10,000 lbs pressure in succession in order to completely form the final tablet. After reducing the pressure on the mold, the tablet was removed, the edges were deburred, and the final tablet weight was recorded. The tablet was then placed in a clean vial, which was subjected to vacuum and purged with nitrogen before the lid was sealed for storage.

Coating Tablets with Hydroxypropyl Cellulose

All tablets were coated by dipping in a 5% solution of hydroxypropyl cellulose (HPC) in isopropyl alcohol. Tablets were held with forceps and dipped by hand into the coating solution. Multiple dips were required to achieve approximately 1.5% coating, and between each dip the tablet was allowed to dry for at least 30 minutes in front of a fan under a fume hood.

The release profile was determined for the individual drugs as per the method of Example 16 and the respective results are summarized in Table IV.

TABLE IV
Release Data of Three-drug Component from Two Layer Tablet II-A*

| Acetaminophen | | | | | Naproxen Sodium | | | | | Ranitidine | | | | |
|---|---|---|---|---|---|---|---|---|---|---|---|---|---|---|
| Hour | % Release | | | Avg. | Hour | % Release | | | Avg. | Hour | % Release | | | Avg. |
| 1 | 8.25 | 8.01 | 6.84 | 7.70 | 1 | 0.26 | 0.18 | 0.26 | 0.23 | 1 | 3.31 | 2.74 | 2.62 | 2.89 |
| 2 | 19.01 | 20.30 | 15.40 | 18.24 | 2 | 50.99 | 56.26 | 57.41 | 54.89 | 2 | 65.20 | 69.65 | 72.64 | 69.16 |
| 3 | 34.13 | 34.59 | 26.37 | 31.70 | 3 | 68.16 | 68.35 | 68.79 | 68.43 | 3 | 85.05 | 84.09 | 85.95 | 85.03 |
| 4 | 47.01 | 45.91 | 36.24 | 43.05 | 4 | 72.18 | 70.83 | 70.80 | 71.27 | 4 | 89.43 | 86.85 | 88.81 | 88.36 |
| 5 | 59.44 | 56.19 | 45.45 | 53.69 | 5 | 73.73 | 72.26 | 71.56 | 72.52 | 5 | 91.37 | 88.05 | 89.94 | 89.79 |
| 6 | 71.24 | 64.37 | 60.49 | 65.70 | 6 | 74.68 | 73.19 | 72.09 | 73.32 | 6 | 92.62 | 88.90 | 90.53 | 90.68 |
| 7 | 81.19 | 74.90 | 70.71 | 75.60 | 7 | 75.29 | 74.04 | 72.42 | 73.92 | 7 | 93.43 | 89.82 | 90.92 | 91.39 |
| 8 | 90.97 | 83.74 | 86.18 | 86.96 | 8 | 75.73 | 74.62 | 72.92 | 74.42 | 8 | 94.09 | 90.58 | 91.84 | 92.17 |

*Coating add-on = 1.60%; data are based on triplicate runs.

Preferred embodiments of the invention have been described using specific terms and devices. The words and terms used are for illustrative purposes only. The words and terms are words and terms of description, rather than of limitation. It is to be understood that changes and variations may be made by those of ordinary skill art without departing from the spirit or scope of the invention, which is set forth in the following claims. In addition it should be understood that aspects of the various embodiments may be interchanged in whole or in part. Therefore, the spirit and scope of the appended claims should not be limited to descriptions and examples herein.

What is claimed is:

1. A combination therapy coated tablet, comprising:
(a) a therapeutically effective dose of a non-steroidal anti-inflammatory drug (NSAID);
(b) a therapeutically effective dose of an agent for reducing gastric acid secretion;
(c) a thermoplastic hydrophilic polymer;
(d) a microparticular excipient; and
(e) a cellulosic enteric coating.

2. A combination therapy coated tablet as in claim 1 wherein the NSAID is selected from the group consisting of naproxen sodium, ibuprofen, and tolmetin, the agent for reducing gastric acid secretion comprises ranitidine, the thermoplastic hydrophilic polymer comprises a solid polyethylene glycol, the microparticular excipient comprises microcrystalline cellulose, and the enteric coating comprises hydroxypropyl cellulose.

3. A combination therapy coated tablet, comprising:
(a) a therapeutically effective dose of an analgesic drug;
(b) a therapeutically effective dose of an agent for reducing gastric acid secretion;
(c) a thermoplastic hydrophilic polymer;
(d) a microparticular excipient; and
(e) a cellulosic enteric coating.

4. A combination therapy coated tablet as in claim 3 wherein the analgesic drug comprises aspirin, the agent for reducing gastric acid secretion comprises omeprazole, the thermoplastic hydrophilic polymer comprises a solid polyethylene glycol, the microparticular excipient comprises microcrystalline cellulose, and the enteric coating comprises hydroxypropyl methyl cellulose phthalate.

5. A combination therapy coated bilayer tablet comprising:
(a) a first layer comprising a therapeutically effective dose of acetaminophen dispersed in a water-soluble thermoplastic polymer; the first layer providing for the delayed release of the acetaminophen;
(b) a second layer comprising a therapeutically effective dose of a non-steroidal anti-inflammatory drug and a therapeutically effective dose of an agent for reducing gastric acid secretion, the second layer providing for the fast release of the non-steroidal anti-inflammatory drug and the agent for reducing gastric acid secretion; and
(c) a polymeric enteric coating.

6. A combination therapy coated bilayer tablet as in claim 5 wherein the water-soluble thermoplastic polymer comprises a solid polyethylene glycol, the non-steroidal anti-inflammatory drug comprises naproxen sodium, the agent for reducing gastric acid secretion comprises ranitidine and the enteric coating comprises hydroxypropyl cellulose.

7. A method for the preparation of the bilayer tablet as in claim 6 comprising the steps of:
(a) dispersing acetaminophen in molten polyethylene glycol;
(b) cooling the acetaminophen/polyethylene glycol system and grinding;
(c) pressing the ground acetaminophen/polyethylene glycol system at room temperature and a pressure of less than 500 lbs;
(d) opening the mold and placing a mixture of the non-steroidal anti-inflammatory drug and the agent for reducing gastric secretion over the formed acetaminophen/polyethylene glycol first layer;
(e) pressing the combined system at room temperature and a pressure of 500 to 1000 lbs;
(f) removing the bilayer tablet from the mold;
(g) dip-coating it in a solution of hydroxypropyl cellulose in 2-propanol; and
(h) allowing the coated tablet to air dry.

8. A combination therapy coated bilayer tablet comprising:
a first layer comprising a therapeutically effective dose of an analgesic drug and a second layer comprising a therapeutically effective dose of an agent for reducing gastric acid secretion wherein each layer contains microparticular polymeric excipients and hydrophilic polymeric excipients.

9. A combination therapy coated bilayer tablet as in claim 8 wherein the analgesic drug comprises aspirin, the acid reducing agent comprises omeprazole, the hydrophilic polymeric excipient comprises a solid polyethylene glycol, and the microparticular excipient comprises microcrystalline cellulose, and further wherein the entire tablet is coated with a hydroxypropyl methyl cellulose phthalate.

10. A combination therapy coated trilayer tablet comprising:
a first layer comprising a therapeutically effective dose of an analgesic drug, microparticular polymeric excipients, and hydrophilic polymeric excipients, a second layer comprising a therapeutically effective dose of an agent for reducing gastric acid secretion, microparticular polymeric excipients, and hydrophilic polymeric excipients, and a third, drug-free layer of a solid, water-soluble polymer adjoining the first and second drug-containing layers.

11. A combination therapy coated trilayer tablet as in claim 10 wherein the analgesic drug comprises aspirin, the agent for reducing gastric acid secretion comprises omeprazole, the microparticular excipient comprises microcrystalline cellulose, the hydrophilic polymeric excipient comprises a polyethylene glycol solid, and the water-soluble polymer comprises a polyethylene glycol, and further wherein the entire tablet is coated with a hydroxypropyl methyl cellulose phthalate.

12. A combination therapy coated trilayer tablet as in claim 11 wherein the agent for reducing gastric acid secretion comprises the magnesium salt of omeprazole.

13. A combination therapy coated trilayer tablet comprising:
a first layer comprising a therapeutically effective dose of an analgesic drug mixed with microparticular and hydrophilic polymeric excipients, a second layer comprising a mixture of microparticular and hydrophilic polymeric excipients, an inorganic base and a therapeutically effective dose of an agent for reducing gastric acid secretion and a third drug-free layer of a solid water-soluble polymer adjoining the first and second drug-containing layers.

14. A combination therapy coated trilayer tablet as in claim 13 wherein the analgesic drug comprises aspirin, the agent for reducing acid secretion comprises omeprazole, the microparticular excipient comprises microcrystalline cellulose, the hydrophilic excipient comprises a polyethylene glycol, the solid water-soluble polymer comprises a polyethylene glycol, and the inorganic base comprises sodium bicarbonate, and further wherein the entire tablet is coated with a hydroxypropyl methyl cellulose phthalate.
